(19) United States
(12) Patent Application Publication
(10) Pub. No.: US 2024/0197733 A1
(43) Pub. Date: Jun. 20, 2024

(54) A GABA RECEPTOR MODULATOR FOR USE IN THE TREATMENT OF PAIN

(71) Applicant: SANIONA A/S, Glostrup (DK)

(72) Inventors: Karin Sandager NIELSEN, Glostrup (DK); Dipak AMRUTKAR, Glostrup (DK); Dean CARSON, Waltham, MA (US)

(21) Appl. No.: 18/554,824

(22) PCT Filed: Apr. 13, 2022
(86) PCT No.: PCT/EP2022/059883

§ 371 Date: Oct. 11, 2023

Related U.S. Application Data
(60) Provisional application No. 63/175,728, filed on Apr. 16, 2021.

(51) Int. Cl. A61K 31/506; A61P 25/02

(52) U.S. Cl. CPC A61K 31/506; A61P 25/02

(57) ABSTRACT

The present invention relates to the use of 2-(3-(3-(2,4-dimethoxypyrimidin-5-yl)phenyl)-3H-imidazo[4,5-b]pyridin-6-yl)propan-2-ol or a pharmaceutically acceptable salt thereof, which is a known GABA receptor modulator, for the treatment of disorders characterized by severe pain, such as trigeminal neuralgia and postoperative pain.

### TECHNICAL FIELD

The present invention relates to the use of 2-(3-(3-(2,4-dimethoxypyrimidin-5-yl)phenyl)-3H-imidazo[4,5-b]pyridin-6-yl)propan-2-ol or a pharmaceutically acceptable salt thereof, which is a known GABA receptor modulator, for the treatment of disorders characterized by severe pain, such as trigeminal neuralgia and postoperative pain.

### BACKGROUND

GABA is the main inhibitory neurotransmitter in the CNS including lamina-II of the spinal cord dorsal horn, where nociceptive fibres terminate. The inhibitory neurotransmission in the spinal cord is of great importance in pain transmission and enhancement of inhibition leads to analgesia (Zeilhofer H U. et. al. (2009), Trends in pharmacological science, 30, 8, 397-402).

Modulators of GABA_{A} receptors have been found to mediate profound analgesia in animal models of neuropathic pain (Munro, G. et al. (2013) European Journal of Pharmacology, 716, 1-3, 17-23). Current therapies for the management of neuropathic pain are of limited benefit to many patients, and involve undesirable side effects or dose-limiting toxicities. In addition, current therapies are symptomatic, not disease modifying. Needs remain for improved therapies for the management and treatment of neuropathic pain, especially those that have the capacity to modify the disease.

The GABA_{A} receptors are ligand gated channels which exists in multiple isoforms. Each receptor is a pentameric complex comprising subunits drawn from α₁₋₆, β₁₋₃, γ₁₋₃, δ, ϵ and θ subunit isoforms. The majority of GABA_{A} receptors present in the CNS contain two α, two β, and one γ subunit (Mckernan R M. et. al., (1996) Trends in Neuroscience, 19, 139-43). The pharmacological effects of activating a GABA_{A} receptor depend mainly on which type of subunits the receptor contains. The classical anxiolytic benzodiazepines show no subtype selectivity. It has been suggested that one of the key elements in the disadvantages of the classical benzodiazepines (such as sedation, dependency, and cognitive impairment) relates to the α₁ and α₅ subunit of the GABA_{A} receptor. Recent studies using mice with point mutations rendering the different α subunits insensitive to diazepam, suggest that α₂ and α₃ subunits mediate the analgesic effects of benzodiazepines (Knabl J. et al., (2009), Pain, 141, 233-38). This is supported by pharmacological studies showing analgesic effects of selective positive modulators of α_{2/3} containing GABA_{A} receptors in preclinical pain models (Munro G. et. al., (2008), JPET, 327, 969-81). Thus, compounds with selectivity for the α₂ and/or α₃ subunits over the α₁ and α₅ subunits are expected to have an improved side effect profile.

WO 2020/053377 discloses 2-(3-(3-(2,4-dimethoxypyrimidin-5-yl)phenyl)-3H-imidazo[4,5-b]pyridin-6-yl)propan-2-ol and its capability of modulating the GABA_{A} receptor complex, and demonstrates that said compound is useful in the treatment of neuropathic pain.

### Trigeminal Neuralgia

Orofacial pain disorders are highly prevalent and debilitating conditions involving head, face and neck. Orofacial pain is caused by musculoskeletal disorders such as temporomandibular disorders (TMD) and neuropathological diseases such as trigeminal neuralgia (TN) (Romero-Reyes M and Uyanik J M, 2014). Trigeminal neuralgia is a disorder of the trigeminal sensory system. It is described as a constant excruciating, sporadic and sudden burning facial pain that lasts from a few seconds up to two minutes per episode. The attacks occur in succession and can last up to two hours. The intense flashes of pain can be triggered by vibration or contact with the cheek (shaving, washing the face, or applying makeup), brushing teeth, eating, drinking, talking, or being exposed to the wind (Maarbjerg S et al., 2017). TN can be caused by mechanical suppression of the trigeminal nerve by cerebral blood vessels resulting in damage to myeline sheets, by tumors and multiple sclerosis or occur sporadically (Bendtsen L et al., 2020). TN pain represents a real challenge to therapy because commonly used drugs are devoid of real benefit and accompanied by side effects or patients become refractory to the treatments (Obermann M, 2019). The first-line treatment of trigeminal neuralgia (TN) is administration of the anticonvulsants carbamazepine or oxcarbazepine, however side-effects are frequently experienced which necessitates drug withdrawal. Consequently, there is an urgent need in the field for provision of improved treatments of trigeminal neuralgia (TN), both curative and palliative.

### Postoperative Pain

Acute and/or chronic postoperative pain originate from damaged and/or dysfunctional nervous tissue as a result of surgery. Very often, medical procedures involving surgery necessitates that nerves are cut during operating procedures, both inadvertently and not, which may result in severe postoperative pain. More often than not, such postoperative pain is ameliorated by administration of strong analgesics such as opioids, which unfortunately in a large number of cases ultimately has been to the detriment of the patient, as drug abuse and drug addiction is a known problem in pain suppression with opioids. Consequently, a need also exists for providing treatment of postoperative pains which does not include administration of highly addictive substances such as opioids or similar, and wherein the dosage is preferably as little as possible.

### SUMMARY

The invention is as defined in the claims.

The present inventors have surprisingly found that acute treatment with 2-(3-(3-(2,4-dimethoxypyrimidin-5-yl)phenyl)-3H-imidazo[4,5-b]pyridin-6-yl)propan-2-ol (compound 1), which is a known GABA_{A} receptor modulator, has an analgesic effect, which compares to traditionally utilized anticonvulsants such as Carbamazepine, but without having the associated adverse effects.

Thus, in a major aspect, the present invention concerns compound 1, which is 2-(3-(3-(2,4-dimethoxypyrimidin-5-yl)phenyl)-3H-imidazo[4,5-b]pyridin-6-yl)propan-2-ol (depicted in formula 1) or a pharmaceutically acceptable salt thereof, for use in the treatment of trigeminal neuralgia (TN). In particular, compound 1 may be included in treatments of orofacial pain in patients suffering from trigeminal neuralgia.

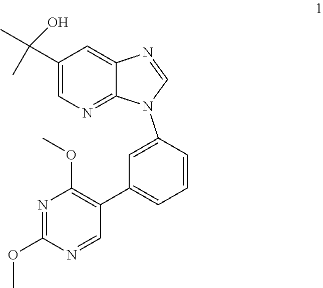 [SMILES: COc1ncc(-c2cccc(-n3cnc4cc(C(C)(C)O)cnc43)c2)c(OC)n1]

In a second aspect, the present invention concerns compound 1 as defined herein for use in the treatment of facial pain in patients suffering from headache and/or migraine. The present inventors have also surprisingly found that chronic/prophylactic treatment with Compound 1 of the present invention, as a once-a-day dose for 7 days is associated with build-up of nociceptive tolerance, evidenced by a reduced induced hyperalgesia in the tested animals subjected to a pain model for facial pain, including headache, migraine and trigeminal neuralgia.

In a third aspect, the present invention concerns compound 1 as defined herein for use in the treatment of postoperative pain, such as acute and/or chronic postoperative pain, originating from surgery (and/or trauma) to the human body.

### DESCRIPTION OF DRAWINGS

FIG. 1: Effect of acute treatment of Compound 1 (1, 3, and 10 mg/kg, po) and Carbamazepine (30 mg/kg, po) on mechanical allodynia in the ES-induced facial pain model. The mechanical pain threshold was evaluated by using Von Frey filaments. Data are shown as mechanical threshold in g plotted on Y-axis, and time on X-axis as baseline (prior to ES) and 0, 2, and 4 hours (post vehicle/compounds treatment). Circle represent vehicle treated rats, rhombus represents carbamazepine treated rats while the square, upward triangle and downward triangle represents the mechanical threshold of rats treated with Compound 1 in 1, 3, and 10 mg/kg respectively. Data are shown as means±S.E.Ms. ####p<0.0001 vs baseline, and */***/**** corresponds to p<0.05/0.001/0.0001 versus vehicle treatments.

FIG. 2: Effect of acute treatment of Compound 1 (1, 3, and 10 mg/kg, po) and Carbamazepine (30 mg/kg, po) on thermal heat hyperalgesia in the ES-induced facial pain model. Thermal pain threshold was evaluated by using a heat lamp and facial withdrawal latency were noted in seconds. Facial withdrawal latencies are plotted on Y-axis and time is plotted on X-axis, baseline (prior to ES) and 0, 2, and 4 hours (post vehicle/compounds treatment). Circle represent vehicle treated rats, rhombus represents carbamazepine treated rats while the square, upward triangle and downward triangle represents rats treated with 1, 3, and 10 mg/kg of Compound 1 respectively. Data are shown as means±S.E.Ms. ####p<0.0001 vs baseline, */**/**** corresponds to p<0.05/0.01/0.0001 versus vehicle treatments.

FIG. 3: Effect of acute treatment with Compound 1 (1, 3, and 10 mg/kg, po) and Carbamazepine (30 mg/kg, po) on thermal cold allodynia in the ES-induced facial pain model. Thermal cold pain threshold was evaluated by applying a drop of acetone to the affected cheek and assessing facial withdrawal latency in seconds. Facial withdrawal latencies in seconds are plotted on Y-axis and time is plotted on X-axis, baseline (prior to ES) and 0, 2, and 4 hours (post vehicle/compounds treatment). Circle represent vehicle treated rats, rhombus represents carbamazepine treated rats while the square, upward triangle and downward triangle represents rats treated with 1, 3, and 10 mg/kg of Compound 1 respectively. Data are shown as mean±S.E.Ms. ####p<0.0001 vs baseline, **/***/**** corresponds to p<0.01/0.001/0.0001 versus vehicle treatments.

FIG. 4: Effect of prophylactic and acute treatment of Compound 1 on mechanical allodynia in the ES-induced facial pain model. The mechanical pain threshold was evaluated by using Von Frey filaments. Data are shown as mechanical threshold in ‘g’ plotted on Y-axis, and time on X-axis as baseline (prior to ES, avg of day 1 and 2) and 0, 2, and 4 hours (post vehicle/compounds treatment, on day 9). Circle represent vehicle treated rats, rhombus represents acute treatment with 3 mg/kg of Compound 1 while the square, upward triangle and downward triangle represents sub-chronically treated rats with 0.3, 1, and 3 mg/kg of Compound 1 respectively. Data are shown as mean±S.E.Ms. */***/**** corresponds to p<0.05/0.001/0.0001 versus vehicle treatments.

FIG. 5: Effect of prophylactic and acute treatment of Compound 1 on thermal hyperalgesia in the ES-induced facial pain model. Thermal pain threshold was evaluated by using a heat lamp and facial withdrawal latency were noted in seconds. Facial withdrawal latencies are plotted on Y-axis and time is plotted on X-axis, baseline (prior to ES, avg on day 1 and 2) and 0, 2, and 4 hours (post vehicle/compounds treatment, on day 9). Circle represent vehicle treated rats, rhombus represents acute treatment with 3 mg/kg of Compound 1 while the square, upward triangle and downward triangle represents sub-chronically treated rats with 0.3, 1, and 3 mg/kg of Compound 1 respectively. Data are shown as mean±S.E.Ms. */**/**** corresponds to p<0.05/0.01/0.0001 versus vehicle treatments.

FIG. 6: Effect of prophylactic and acute treatment of Compound 1 on thermal cold allodynia in the ES-induced facial pain model. Thermal cold pain threshold was evaluated by applying a drop of acetone to the affected cheek and assessing facial withdrawal latency in seconds. Facial withdrawal latencies in seconds are plotted on Y-axis and time is plotted on X-axis, baseline (prior to ES. Avg on day 1 and 2) and 0, 2, and 4 hours (post vehicle/compounds treatment, on day 9). Circle represent vehicle treated rats, rhombus represents acute treatment with 3 mg/kg of Compound 1 while the square, upward triangle and downward triangle represents sub-chronically treated rats with 0.3, 1, and 3 mg/kg of Compound 1 respectively. Data are shown as mean±S.E.Ms. **/***/**** corresponds to p<0.01/0.001/0.0001 versus vehicle treatments.

### DETAILED DESCRIPTION

The trigeminal nerve, also known as the fifth cranial nerve, is a nerve responsible for sensation in the face and motor functions such as biting and chewing. This large nerve largely consists of three major branches; the ophthalmic nerve (V₁), the maxillary nerve (V₂), and the mandibular nerve (V₃).

Trigeminal neuralgia (TN) is a medical condition associated with hypersensitivity of one or more of the trigeminal nerve branches, resulting in a painful sensation of the facial area of affected patients. The pain may be double- or one-sided. Likewise, the pain may be constant or periodic or both; the duration of periodic pain attacks varying from a few seconds to several minutes or even hours. Common everyday activities such as eating, talking, shaving and brushing teeth may trigger attacks which can lead to secondary conditions such as neuropathic and/or muscular headaches and migraines. Patients affected by trigeminal neuralgia (TN) describe the attacks as feeling like stabbing electric shocks, burning, sharp, pressing, crushing, exploding or shooting pain. The exact cause of the condition is unknown but believed to involve loss of myelin of the trigeminal nerve.

### Biological Activity

Compound 1 of the present invention is capable of modulation of the GABA_{A} receptor complex, and is a proven positive allosteric modulator (PAM) of GABA_{A} receptors containing the a₃ subunit and, to a minor extent, a₂ and a₅ subunits. Compound 1 is demonstrated herein to reverse hyperexcitability produced by electrical stimulation in a rat model for trigeminal neuralgia after acute treatment. Compound 1 is further demonstrated to have an increased analgesic effect if administered prophylactically such as a once daily dosage for 7 days prior to the insult.

Animal models help in understanding the pathogenesis of diseases and in finding new treatment options. Research in trigeminal neuralgia is hampered by the limited number of available animal models which are infraorbital nerve chronic constriction injury model (ION-CCI) and ligation of infraorbital ligation (Luo D S et al., 2012). The electrocutaneous stimulation-induced facial pain rodent model is a surgery-free reproducible orofacial pain model in which a depilated cheek area, of rats, comprising the receptive field of the trigeminal sensory nerve, is repeatedly subjected to electrical stimulation. Repeated electrical stimulation at low intensities produces craniofacial pain that mimics the behavioral characteristics of trigeminal neuralgia such as development of mechanical allodynia and thermal hyperalgesia at the cheeks despite no visible edema, swelling or excessive injury and tissue damage.

### Methods of Therapy

Being a ligand for GABA_{A} receptors, Compound 1 is of use in the treatment, prevention, and/or alleviation of disorders of a living body, including human. In one embodiment of the present invention, the treatment by Compound 1 is an antinociceptive treatment of pain, such as facial pain, orofacial pain and craniofacial pain.

In one embodiment of the present invention, the antinociceptive treatment is preemptive analgesia.

In one aspect, the present invention relates to 2-(3-(3-(2,4-dimethoxypyrimidin-5-yl)phenyl)-3H-imidazo[4,5-b]pyridin-6-yl)propan-2-ol, or a pharmaceutically acceptable salt thereof, for use in the treatment of facial pain.

In one aspect, the present invention relates to 2-(3-(3-(2,4-dimethoxypyrimidin-5-yl)phenyl)-3H-imidazo[4,5-b]pyridin-6-yl)propan-2-ol, or a pharmaceutically acceptable salt thereof, for use in the treatment of postoperative pain.

In one aspect, the present invention relates to a method for treatment of facial pain and/or postoperative pain comprising administration of 2-(3-(3-(2,4-dimethoxypyrimidin-5-yl)phenyl)-3H-imidazo[4,5-b]pyridin-6-yl)propan-2-ol, or a pharmaceutically acceptable salt thereof, to a subject in need thereof.

In one aspect, the present invention relates to use of 2-(3-(3-(2,4-dimethoxypyrimidin-5-yl)phenyl)-3H-imidazo[4,5-b]pyridin-6-yl)propan-2-ol, or a pharmaceutically acceptable salt thereof, for the manufacture of a medicament for the treatment of facial pain and/or postoperative pain.

In one embodiment, the facial pain is orofacial pain and/or craniofacial pain.

In one embodiment, the facial pain is associated with migraine or headache. In one embodiment, the pain is associated with atypical facial pain or mononeuropathies.

In one embodiment of the present invention, the treatment by Compound 1 is acute. In one embodiment of the present invention, the treatment by Compound 1 is prophylactic. In one embodiment of the present invention, the treatment by Compound 1 is preventive. In one embodiment of the present invention, the treatment by Compound 1 is ameliorative. In one embodiment of the present invention, the treatment by Compound 1 is symptomatic.

In one embodiment of the present invention, the treatment by Compound 1 is administered perorally to a subject as a dose ranging from 0.001 mg/kg-100 mg/kg, such as 0.001-0.01 mg/kg, such as 0.01-0.05 mg/kg, such as 0.05-1.0 mg/kg, such as 1-5 mg/kg, such as 5-10 mg/kg, such as 10-25 mg/kg, such as 25-50 mg/kg, such as 50-100 mg/kg.

In one embodiment of the present invention, the treatment by Compound 1 is administered to a subject intravenously as a dose ranging from 0.1 μg/kg-10 mg/kg, such as 0.1-1 μg/kg, such as 1.0-10 μg/kg, such as 10-50 μg/kg, such as 50 μg/kg-1.0 mg/kg, such as 0.1-0.5 mg/kg, such as 0.5-1.0 mg/kg, such as 1.0-2.5 mg/kg, such as 2.5-5.0 mg/kg, such as 5.0-10 mg/kg.

In one embodiment, compound 1 is administered in combination with one or more additional therapeutically active ingredient.

### Treatment of Trigeminal Neuralgia (TN) (ICD-11 8B82.0)

In one embodiment, the facial pain is trigeminal neuralgia (TN). Trigeminal neuralgia (TN) is a medical condition associated with hypersensitivity of one or more of the trigeminal nerve branches, resulting in a painful sensation of the facial area of affected patients. The pain may be double- or one-sided. Likewise, the pain may be constant or periodic or both; the duration of periodic pain attacks varying from a few seconds to several minutes or even hours. The exact cause of the condition is unknown but believed to involve loss of myelin of the trigeminal nerve.

Preferably, Compound 1 is for use in the treatment, prevention, and/or alleviation of pain, such as associated with pain in subjects suffering from trigeminal neuralgia or other facial pain disorders such as headache and/or migraine. In one embodiment, the present invention relates to a method for relieving symptoms of trigeminal neuralgia in a patient.

In one embodiment, Compound 1 is for use in the treatment, prevention, and/or alleviation of facial pain, such as orofacial pain, in patients suffering from headache and/or migraine. Exemplary headache- and migraine-related conditions as defined by ICD-11 MMS (ICD-11 for Mortality and Morbidity Statistics) particularly suited for treatment by use of the invention may be migraine (8A80), migraine without aura (8A80.0), migraine with aura (8A80.1), chronic migraine (8A80.2), tension type headache (8A81), infrequent episodic tension-type headache (8A81.0), frequent episodic tension-type headache (8A81.1), chronic tension-type headache (8A81.2), trigeminal autonomic cephalalgias (8A82), and painful cranial neuropathies or other facial pains (8A85). In one embodiment, the present invention relates to Compound 1 for use in the treatment, prevention, and/or alleviation of facial pain, such as orofacial pain, in patients suffering from headache and/or migraine, wherein said headache and/or migraine is of neuropathic and/or muscular character. In one embodiment, Compound 1 is for use in the treatment, prevention, and/or alleviation of headache and/or migraine.

In one embodiment, Compound 1 is for use in the treatment, prevention, and/or alleviation of disorders of the trigeminal nerve, such as defined by ICD-11 MMS (ICD-11 for Mortality and Morbidity Statistics), i.e. the disorders of the trigeminal nerve (8B82) are selected from trigeminal neuralgia [8B82.0], atypical facial pain [8B82.1], and unspecified disorders of the trigeminal nerve [8B82.Z]. The references in brackets refer to the ICD-11 nomenclature. In one embodiment, the present invention relates to Compound 1 for use in the treatment of pain associated with trigeminal neuralgia, mononeuropathies or/and atypical facial pain.

As disclosed herein, the disorder “trigeminal neuralgia” may be interpreted as a genus covering other disorders related to the function of the trigeminal nerve. Such subdisorders may be one or more of classical trigeminal neuralgia, secondary trigeminal neuralgia, idiopathic trigeminal neuralgia and painful trigeminal neuropathy. Each of the disorders mentioned in the previous section may in addition cover multiple disorders, such as found in the ICHD-3 (International Classification of Headache Disorders, 3^{rd} edition 2018).

In a preferred embodiment of the present invention, a pharmaceutical composition comprising Compound 1, or a pharmaceutically acceptable salt thereof, is for use in the treatment of trigeminal neuralgia (TN).

In another embodiment, the use of the provided composition for treatment of trigeminal neuralgia is curative, preventive, protective, palliative, prophylactic, symptomatic, and/or ameliorative.

Another embodiment of the present invention is a method for treatment of trigeminal neuralgia (TN) comprising administration of a composition comprising Compound 1, or a pharmaceutically acceptable salt thereof, to a subject in need thereof. In one embodiment of the present invention, the pharmaceutical composition comprises an amount of Compound 1, or a pharmaceutically acceptable salt thereof, that is effective in treating TN in a subject.

One embodiment of the present invention is use of Compound 1, or a pharmaceutically acceptable salt thereof, in the manufacture of a medicament for treatment of trigeminal neuralgia (TN).

One embodiment of the present invention is use of a composition comprising Compound 1, or a pharmaceutically acceptable salt thereof, in the manufacture of a medicament for treatment of trigeminal neuralgia (TN).

### Treatment of Acute Postoperative Pain (ICD-11 MG31.2)

In one embodiment, the postoperative pain is acute postoperative pain. Acute postoperative pain is classified as pain at the intervention site or caused by an intervention such as a surgical procedure with an expected pain duration of less than 3 months. Preferably, Compound 1, or a pharmaceutically acceptable salt thereof, is for use in the treatment, prevention, and/or alleviation of pain, such as associated with pain in subjects suffering from acute postoperative pain.

### Treatment of Chronic Postoperative Pain (ICD-11 MG30.21)

In one embodiment, the postoperative pain is chronic postoperative pain. Chronic postoperative pain is chronic pain developing or increasing in intensity after a surgical procedure and persisting beyond the healing process, i.e. at least 3 months after surgery. The classification includes but is not limited to chronic pain after surgery, such as surgical procedures comprising spinal surgery, herniotomy, hysterectomy, amputation, thoracotomy, breast surgery, and arthroplasty. The pain is either localized to the surgical field, projected to the innervation territory of a nerve situated in this area, or referred to a dermatome (after surgery/injury to deep somatic or visceral tissues). Depending on the type of surgery, the chronic postsurgical pain may also be classified as neuropathic pain.

Preferably, Compound 1, or a pharmaceutically acceptable salt thereof, is for use in the treatment, prevention, and/or alleviation of pain, such as associated with pain in subjects suffering from chronic postoperative pain.

Another embodiment of the present invention is a method for treatment of acute and/or postoperative pain comprising administration of a composition comprising Compound 1, or a pharmaceutically acceptable salt thereof, to a subject in need thereof. In one embodiment of the present invention, the pharmaceutical composition described herein comprises an amount of Compound 1, or a pharmaceutically acceptable salt thereof, that is effective in treating postoperative pain in a subject.

In one embodiment of the present invention, the pharmaceutical composition described herein comprises an amount of Compound 1, or a pharmaceutically acceptable salt thereof, that is effective in treating acute- and/or chronic postoperative pain in a subject.

In one embodiment of the present invention, Compound 1, or a pharmaceutically acceptable salt thereof, is for use in treatment of acute and/or chronic postoperative pain, wherein the treatment is initiated more than 2 days, such as 3, such as 4, such as 5, such as 6, such as 7, such as 10, such as 20, such as 30 days prior to the subject undergoing surgery. In one embodiment of the present invention, Compound 1, or a pharmaceutically acceptable salt thereof, is for use in treatment of acute and/or chronic postoperative pain, wherein the treatment is initiated more than 1 day, such as 2, such as 3, such as 4, such as 5, such as 6, such as 7, such as 14, such as 30, such as 60, such as more than 90 days following the subject undergoing surgery.

### Pharmaceutically Acceptable Salts

The chemical compound of the invention may be provided in any form suitable for the intended administration, including pharmaceutically (i.e. physiologically) acceptable salts. Examples of pharmaceutically acceptable addition salts include, without limitation, non-toxic inorganic and organic acid addition salts such as hydrochloride, hydrobromide, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, salicylate, sorbate, stearate, succinate, tartrate, toluene-p-sulphonate, and the like. Such salts may be formed by procedures well known and described in the art. Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of compound 1 of the invention include, without limitation, the sodium, the potassium, the calcium, the magnesium, the zinc, the aluminium, the lithium, the choline, the lysinium, and the ammonium salt, and the like, of compound 1 of the invention containing an anionic group. Such cationic salts may be formed by procedures well known and described in the art. In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

### Labelled Compounds

The chemical compound of the present invention may be used in its labelled or un-labelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging. The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Pharmaceutical Compositions

The invention also provides use of pharmaceutical compositions comprising therapeutically effective amount of compound 1, or a pharmaceutically acceptable salt thereof, together with at least one pharmaceutically acceptable carrier, excipient or diluent.

While compound 1 of the present invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt thereof, together with one or more pharmaceutically acceptable carriers, and, optionally, other therapeutic and/or prophylactic ingredients, known and used in the art. The carrier(s) should be “acceptable” in the sense of being compatible with the other ingredients of the formulation and not harmful to the recipient thereof. Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

Compound 1 of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Compound 1 of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from compound 1 of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In one embodiment of the present invention, Compound 1, or the pharmaceutically acceptable salt thereof, is the only active pharmaceutical ingredient. In one embodiment of the present invention, Compound 1 is within a pharmaceutical composition, wherein the composition comprises one or more adjuvants, excipients, carriers, buffers, diluents, and/or other pharmaceutical auxiliaries. In one embodiment of the present invention, the pharmaceutical composition consists of Compound 1, or the pharmaceutically acceptable salt thereof, and one or more adjuvants, excipients, carriers, buffers, diluents, and/or other pharmaceutical auxiliaries.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

A therapeutically effective dose refers to that amount of active ingredient, which ameliorates the symptoms or condition. Therapeutic efficacy and toxicity, e.g. ED₅₀, may be determined by standard pharmacological procedures in cell cultures or experimental animals. The dose ratio between therapeutic and toxic effects is the therapeutic index and may be expressed by ratio between plasma levels resulting in therapeutic effects and plasma ratios resulting in toxic effects. Pharmaceutical compositions exhibiting large therapeutic indexes are preferred.

The dose administered may be adjusted to the age, weight and condition of the individual being treated, as well as the route of administration, dosage form and regimen, and the result desired, and the exact dosage can be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 10,000 mg of active ingredient per individual dose, preferably of from about 1 to about 1000 mg, most preferred of from about 10 to about 500 mg, are suitable for therapeutic treatments. The active ingredient may be administered in one or several doses per day. A satisfactory result may, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Definitions

The term “preventive treatment,” as used herein to describe the present invention, means that the compound, pharmaceutical composition or combination is administered to a subject to inhibit or stop or reduce the risk of the relevant condition or symptom of said condition from occurring in a subject.

The term “prophylactic treatment” as used herein to describe the present invention, refers to the treatment of subjects having, or at risk of having a specified condition or symptom of the condition, but not presently having or experiencing the symptoms of the condition.

The term “ameliorative treatment” as used herein to describe the present invention, refers to the treatment of subjects having, or at risk of having a specified symptom, wherein the subjects are presently having or experiencing the symptoms of the condition.

The term “symptomatic treatment” as used herein to describe the present invention, refers to ameliorating a specified condition or disorder or symptoms accompanied therewith to a significant extent.

### EXAMPLES

### Example 1—Acute Treatment of Pain with Compound 1

### Materials and Methods

### Animal Model:

The electrocutaneous stimulation-induced facial pain rodent model is a reproducible orofacial pain model in which depilated cheek area of rats are repeatedly subjected to electrical stimulation. Repeated electrical stimulation at low intensities produces craniofacial pain that mimics the behavioral characteristics of TN such as development of mechanical and thermal allodynia at cheeks despite no visible edema, swelling or excessive injury and tissue damage.

### Animals:

Male wild type, Sprague-Dawley rats (Charles River Laboratories) born in-house at AfaSci Research Laboratory between May 24, 2020 to May 28, 2020. At approximately 3 months of age (approximately 350 g-450 g body weight) the rats were deemed suitable for the electrocutaneous stimulation-induced facial pain model. Prior to the experiment, the rats were maintained in group cages (3 rats per cage) in a controlled environment (temperature: 21.5±4.5° C./relative humidity: 35-55%) under a standard 12 h light/12 h dark lighting cycle (lights on at 06:00).

### Solvents:

Dimethyl sulfoxide (DMSO)

2-hydroxypropyl-beta-cyclodextrin (HPBCD)

### Compounds:

Compound 1, Molecular weight 391.42 g/mol.

### Carbamazepine/Tegretol, Molecular weight 236.27 g/mol.

### Compound 1 and Carbamazepine (CBZ) Preparation

The required quantity of the compounds are weighed and dissolved in 100% DMSO in a volume amounting to 5% of the total volume while placed on a stirrer. The weight of the compounds refers to the free base weights. Once the solution becomes clear/transparent, the remaining volume of the vehicle is added (30% HPBCD prepared in MQ water) to final concentrations of 0.1, 0.3, and 1 mg/ml for Compound 1 and 3 mg/ml of Carbamazepine. The solutions are kept on a stirrer before- and during administration of the compounds to the animals.

Route of administration: perorally (po)

Dose volume: 10 ml/kg

Treatment Groups (final number of rats after baseline measurements for mechanical and thermal hypersensitivity): N=8, per group

The experimenter assessing the behavioral responses to drug administration is blinded to the drug treatment to the animals. Each treatment group is coded according the scheme below:
A. Vehicle (5% DMSO+30% HPBCD): 10 ml/kg
B. Compound 1: 1 mg/kg
C. Compound 1: 3 mg/kg
D. Compound 1: 10 mg/kg
F. Carbamazepine: 30 mg/kg

### Timeline of Experimental Design:

### Day 0, Habituation:

All rats were allowed to accommodate to the decapicone restraint for 15 minutes each

### Day 1, 1^{st} Baseline Measurement:

Rats were allowed to accommodate to the pain testing apparatus (Von Frey mesh for mechanical pain) and decapicone restraint (for heat- and cold stimulation) for 15 minutes each prior to baseline nociceptive measurements using mechanical- (Von Frey filaments), heat- (heat lamp) and cold- (˜10 μl drop of acetone) stimulation (for description of the separate pain tests, see below). The tests were carried out in this test order.

### Day 2, 2^{nd} Baseline Measurement and Electrocutaneous Stimulation (ES):

Second baseline measurement was carried out similarly to day 1. Immediately following the second baseline recording, each rat received 1 hr of electrocutaneous stimulation at cheek (0.4 mA intensity, 10 ms duration, 1 s inter-pulse interval). For each animal, the average of the two baseline nociceptive measurements were calculated within each pain modality (mechanical, heat- and cold pain), and used as baseline (“Baseline” point on figures).

Day 3, Assessment of ES Induced Facial Mechanical and Thermal (Heat- and Cold) Allodynia and Effect of Acute Treatment with Compound 1 and Carbamazepine:

Similarly, to day 1- and 2, all rats were subjected to acclimatization to test equipment and decapicone followed by measurements of ES-induced facial mechanical- heat- and cold allodynia (“0” time point on figures). Based on the individual rat's mechanical- and thermal hypersensitivity, 40 rats were selected for drug testing. Compound 1, vehicle and Carbamazepine was administered perorally in doses of 1, 3, and 10 mg/kg (Compound 1) or 30 mg/kg (carbamazepine) and drug effects on pain thresholds were evaluated 2- and 4 hours after drug administration by assessment of mechanical- (von Frey) and thermal hypersensitivity (heat- and cold stimulation respectively).

Terminal blood was sampled and prepared (plasma) immediately after the last timepoint. Brains were collected and snap frozen on dry ice (without perfusion). Plasma and tissue was stored at −20.

50 rats were included at start of the experiment and 10 rats were stimulated each day. Each set of 10 rats were dosed and tested 24 hours after their electrocutaneous stimulation (assuming each rat was significantly hypersensitive prior to dosing based on mechanical and thermal pain assessments, pain baseline/“0 hours”). This procedure of dosing and testing 10 rats was repeated each day so that a total number of 40 rats were included in the drug testing.

Body weights of all rats were recorded for accuracy of proper dose delivery as well as for health monitoring purposes.

### Behavioral Assessment of Pain

### Baseline- and ES-Induced Mechanical- Heat and Cold Pain Thresholds:

Decrease in ES-induced pain threshold was evaluated 22 hours following electrical stimulation of the depilated left cheek area (between the eye and ear ridge, this is a receptive field of trigeminal nerve) of each rat and was manifested as facial withdrawal following application of Von Frey filaments for mechanical allodynia and a heat lamp and acetone test for thermal hyperalgesia and allodynia respectively. Baseline pain behaviors were measured similarly to ES-induced facial pain measurements, except that rats were not subjected to ES stimulation.

Assessment of nociceptive behaviors (facial withdrawal) was conducted in the following order during baseline measurements, prior to drug treatment and after drug treatment: mechanical- (von Frey filaments), heat- (heat lamp) and cold (acetone) test.

### Mechanical Allodynia (Von Frey Filaments):

Mechanical pain thresholds were assessed by measuring each rat's facial withdrawal threshold (g) in response to von Frey monofilaments (BioSeb). Starting monofilament: 3.84 (0.6 g), 50% facial withdrawal threshold was interpolated using the up-down method (Chaplan S R et al., 1994). Cutoff monofilament was 4.56 (4 g).

### Thermal Hot Hyperalgesia (Lamp Test):

Immediately following assessment of mechanical pain threshold, thermal (hot- and cold induced) pain was determined. Hot thermal pain thresholds were assessed by measuring each rat's facial withdrawal latency (seconds) in response to thermal stimulation on the depilated and stimulated rat cheek by using Yeomans' Thermal Testing Lamp with the following settings: Lamp fixed ˜7 cm above depilated left cheek of rat, Voltage regulator was set to 45V for C fiber testing. Cutoff was set at 20 seconds to avoid tissue damage.

### Cold Thermal Allodynia (Acetone Test):

Cold Thermal threshold was assessed by measuring each rat's facial withdrawal latency (seconds) in response to a single 10 uL drop of acetone directly on the depilated and stimulated rat cheek. Cutoff was set at 60 seconds. Facial withdrawal response criteria were as following: burrowing head, shaking of head back-and-forth, excessive blinking, turning head away from experimenter.

### Statistical Evaluation:

Statistical comparisons of mechanical allodynia-, heat hyperalgesia and cold allodynia, between vehicle treated and drug treated groups were analyzed by two-way ANOVA (time and drug treatment as factors), followed by Fishers LSD test for post hoc comparisons. The level of significance was set at P<0.05. All the analyses were performed using Graph Pad prism 9.0 software (GraphPad Software, San Diego, CA) and data is presented as mean±SEM.

### Example 1a: Mechanical Pain (Von Frey)

Direct application of low intensity electrical stimulation causes significant hypersensitivity in the stimulated area (depilated left cheek). The decrease in the mechanical pain threshold to the Von Frey filaments after ES is referred as mechanical allodynia.

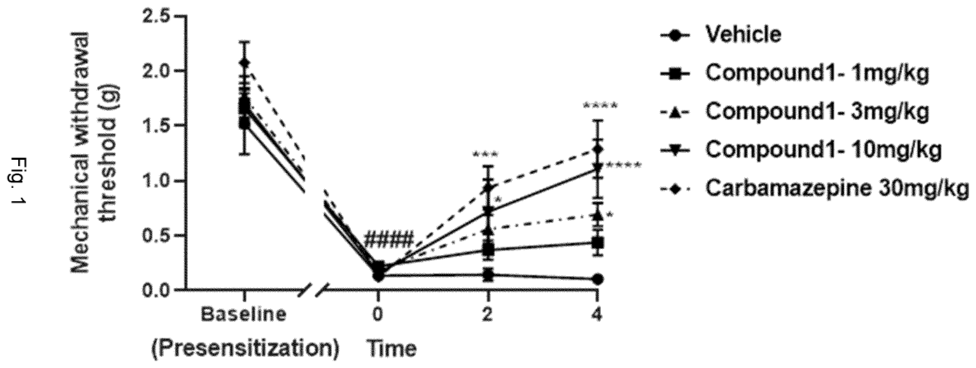

All groups displayed an increase in mechanical hypersensitivity as evidenced by the robust decrease in mechanical pain threshold from baseline (average of Day 1 and Day 2 measurements) to Day 3 (0 time, p<0.0001). As seen in FIG. 1, the results illustrate a direct dose-dependent and significant analgesic effect of Compound 1, at 3- and 10 mg/kg as compared to vehicle treatment at 4 hours post dosing (p<0.05, and p<0.0001 for 3- and 10 mg/kg respectively). Compound 1, 10 mg/kg also reversed the mechanical allodynia at 2 hrs (p<0.05 as compared to vehicle treatment). The standard of care, Carbamazepine resulted in significant analgesic effects at 2- and 4 hours post dosing (p<0.001, p<0.0001 vs vehicle treatment respectively). Highest dose of Compound 1, 10 mg/kg, po., displayed a comparable analgesic effect with carbamazepine 30 mg/kg, p.o post 4 hrs of treatment.

### Example 1b—Hot Thermal Hyperalgesia (Selective C Fiber Testing)

After repeated electrocutaneous stimulation, wide dynamic range neurons show increased excitability. This hyper-excitability can be caused by an increased neuronal response to a noxious stimulus (hyperalgesia). This condition is maintained by C afferent fibers.

C fibers cause central sensitization of the dorsal horn neurons in the spinal cord. The mechanism underlying this phenomenon involves the release of glutamate by these sensitized C fibers, glutamate interacts with the postsynaptic NMDA receptors of the dorsal horn neurons. This central sensitization results in increased pain (hyperalgesia) and pain responses from previously non-noxious stimuli evoke a pain response (allodynia).

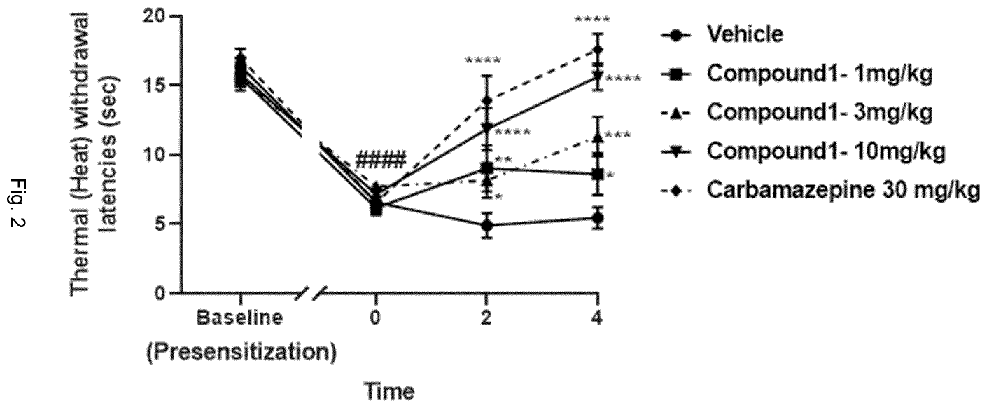

For selective C fiber testing, the rats were lightly restrained in a tipless decapicone and exposed to a noxious heat stimulus; time taken by rat to withdraw its head away from the heat stimulus, was recorded as an index of a hot thermal pain threshold. A naive rat typically can sustain approximately 15-17 seconds of constant heat exposure before it withdraws its cheek from the light source. As seen in FIG. 2, after electrocutaneous stimulated rats developed thermal hypersensitive and showed a considerable drop in cheek withdrawal latency from an average of 16 seconds (baseline) to an average of 6 seconds post-electrocutaneous stimulation/0 hour. The withdrawal latency of all rats was significantly reduced compared to their baseline (p<0.0001). Compound 1 dose dependently reversed the heat thermal hyperalgesia. The minimal effective dose was 1 mg/kg (p<0.01 and p<0.05 compared to vehicle treatment 2- and 4 hrs post dose respectively). Compound 1 at 3 mg/kg significantly reversed the mechanical allodynia post 2 and 4 hrs p<0.05 and p<0.001 vs vehicle respectively. Compound 1 at 10 mg/kg significantly produced reversal of hyperalgesia, as the withdrawal latencies 2 and 4 hrs post dose (p<0.0001). The standard of care, Carbamazepine similarly, showed full reversal of heat hyperalgesia post 4 hours of dose (p<0.0001)

### Example 1c—Cold Thermal Allodynia (Acetone Test)

Acetone evaporation test is widely used to assess the cold sensitivity in animal models of pain. It involves the direct application of acetone (10 uL drop), after a few seconds it evaporates and cools the applied area. The time taken by the rat to begin burrowing, shaking, or withdrawing its head after application of acetone is used as a measure of cold sensitivity. Acetone test is a simple and reproducible, we have used to assess the cold allodynia after electrocutaneous stimulation-induced facial pain.

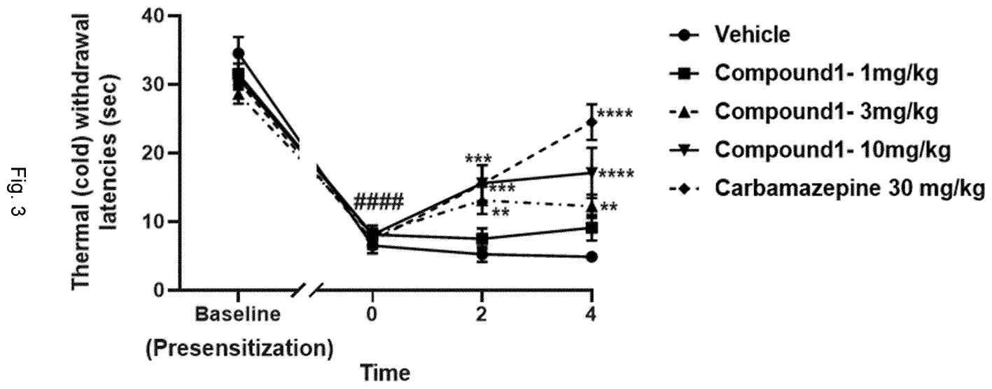

Rats were lightly restrained in a tipless decapicone and their cheeks were exposed to a single 10 uL drop of acetone, time taken by rat to withdraw/shake/burrow its head was recorded as cold thermal pain threshold. A naive rat typically responds approximately within 30-35 seconds after the application of acetone. As seen in FIG. 3, rats subjected to electrocutaneous stimulation developed thermal cold hypersensitivity, they showed significant fall in withdrawal latency from an average of 35 seconds (baseline) to approximately 6 seconds (22 hours post-electrocutaneous stimulation/0 hour). The withdrawal latency of all rats was significantly reduced compared to their baseline values (p<0.0001). Compound 1 reversed the cold allodynia by a minimum effective dose of 3 mg/kg (p<0.01 after 2- and 4 hrs of dosing vs. vehicle). The highest dose of Compound 1, 10 mg/kg, resulted in significant enhancement of the cold pain threshold following 2- and 4 hours of treatment, p<0.001, and p<0.0001 respectively vs vehicle treatment.

Reference standard, Carbamazepine showed significant enhancement of the cold pain threshold at 2 and 4 hrs post dose in the acetone test, p<0.001 and p<0.0001 vs vehicle treatment respectively.

### Conclusion to Example 1

Direct electrical stimulation to depilated cheek area of rats produces the development of allodynia and hyperalgesia manifested as a marked reduction in mechanical and thermal pain thresholds representative of symptoms of trigeminal neuralgia after 22 hours of stimulation, despite no visible edema or swelling.

Acute treatment with the test article Compound 1 (1, 3 and 10 mg/kg, po) produces significant analgesic effects in a dose dependent manner with a minimal effective dose between 1- and 3 mg/kg. The highest dose tested, 10 mg/kg, results in full reversal of hyperalgesia, similar to the standard of care, Carbamazepine 30 mg/kg, in the thermal heat test. These data supports potential effect of Compound 1 for trigeminal neuralgia and other facial neuropathic pain disorders.

### Example 2—Prophylactic Treatment of Pain with Compound 1

### Materials and Methods

Unless stated otherwise, the same materials, methods, and provisions of Example 1 were applied to Example 2.

### Compound 1 Preparation

The required quantity of the compound is weighed and dissolved in 100% DMSO in a volume amounting to 5% of the total volume while placed on a stirrer. The weight of the compounds refers to the free base weights. Once the solution becomes clear/transparent, the remaining volume of the vehicle is added (30% HPBCD prepared in MQ water) to final concentrations of 0.03, 0.1, and 0.3 mg/ml for Compound 1. The solutions are kept on a stirrer before- and during administration of the compounds to the animals.

Route of administration: perorally (po)

Dose volume: 10 ml/kg

Treatment Groups (final number of rats after baseline measurements for mechanical and thermal hypersensitivity): N=8, per group

The experimenter assessing the behavioral responses to drug administration is blinded to the drug treatment to the animals. Each treatment group is coded according the scheme below:
A. Vehicle (5% DMSO+30% HPBCD): 10 ml/kg
B. Compound 1: 0.3 mg/kg
C. Compound 1: 1 mg/kg
D. Compound 1: 3 mg/kg
F. Compound 1: 3 mg/kg—acute

### Timeline of Experimental Design:

### Day 0, Habituation:

All rats were allowed to accommodate to the decapicone restraint for 15 minutes each

### Day 1, 1^{st} Baseline Measurement:

1^{st} baseline measurement: Rats acclimatized to the pain testing apparatus (Von Frey mesh for mechanical pain) and decapicone restraint (for heat- and cold stimulation) for 15 minutes each prior to baseline measurements using mechanical- (Von Frey filaments), heat- (heat lamp) and cold- (˜10 μl drop of acetone) stimulation (for description of the separate pain tests, see below). The tests were carried out in this test order.

### Day 2, 2^{nd} Baseline Measurement and Electrocutaneous Stimulation (ES):

Second baseline measurement was carried out similarly to day 1, For each animal, the average of the two baseline nociceptive measurements was calculated within each pain modality (mechanical, heat- and cold pain), and used as baseline (“Baseline” point on figures). Then all rats in the above respective group (A-D) are given treated as mentioned. Group F received vehicle treatment.

### Day 3-7:

Treatment is continued once daily to each group same as Day 2

### Day 8:

All rats were treated with respective treatment as per day 3-7. After 1 hr of treatment, each rat was subjected to 1 hr of electrocutaneous stimulation at depilated cheek (0.4 mA intensity, 10 ms duration, 1 s inter-pulse interval).

### Day 9, Assessment of ES Induced Facial Mechanical and Thermal (Heat- and Cold) Allodynia and Prophylactic and Acute Effect of Compound 1:

Similar to day 1- and 2, all rats were acclimatized to test equipment followed by measurements of ES-induced facial mechanical- heat- and cold allodynia (“0” time point on figures). After measurement of hypersensitivity threshold, rats from group A-D received the vehicle and three doses of Compound 1 as mentioned above while group F received single acute dose of 3 mg/kg of Compound 1. Treatment effects on pain thresholds were evaluated 2- and 4 hours after administration by assessing mechanical- (Von Frey) and thermal (heat and cold) withdrawal threshold.

Terminal blood was sampled and prepared (plasma) immediately after the last timepoint. Brains were collected and snap froze on dry ice (without perfusion). Plasma and tissue was stored at −20° C.

Body weights of all rats were recorded for accuracy of proper dose delivery as well as for health monitoring purposes.

### Example 2a: Mechanical Pain (Von Frey)

General considerations as given under Example 1a also apply here.

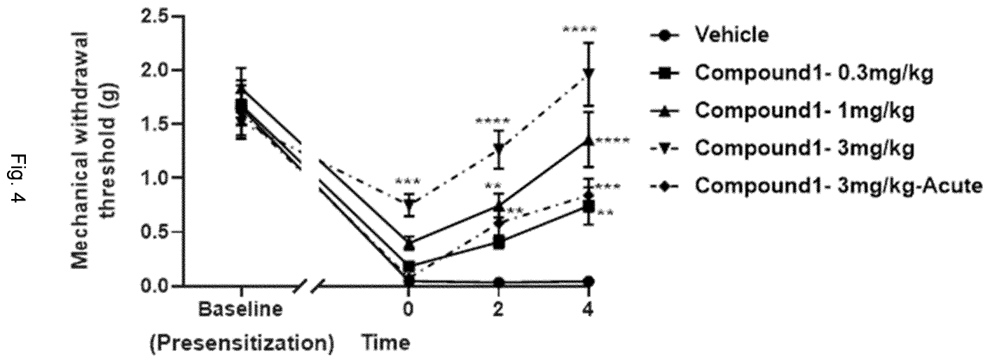

All rats displayed an increase in mechanical hypersensitivity as evidenced by the robust decrease in mechanical pain threshold from baseline (average of Day 1 and Day 2 measurements) to Day 9 (0 time, p<0.0001 vs baseline). As seen in FIG. 4, prophylactic treatment of Compound 1, 3 mg/kg for 7 days significantly reduced the development of allodynia compared to vehicle treatment (p<0.001 vs vehicle at 0 time). After establishment of ES induced mechanical allodynia, additional dose of Compound 1 on day 9, reversed the mechanical allodynia dose dependently, p>0.05, p<0.01 and p<0.0001 post 2 hrs and p<0.01, p<0.0001 and p<0.0001 post 4 hrs for 0.3, 1 and 3 mg/kg respectively. Acute treatment of Compound 1 at 3 mg/kg also reversed the mechanical allodynia, p<0.01, post 2 hrs and p<0.001, post 4 hrs. The effect size is smaller compared to the prophylactic treated animals.

### Example 2b—Hot Thermal Hyperalgesia (Selective C Fiber Testing)

General considerations as given under Example 1b also apply here.

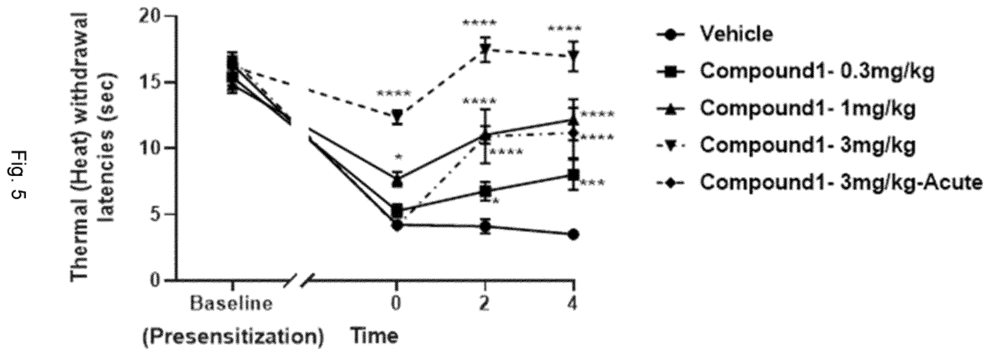

All rats displayed an increase in heat hyperalgesia as evidenced by the robust decrease in heat pain threshold from baseline (average of Day 1 and Day 2 measurements) to Day 9 (0 time, p<0.0001 vs baseline). As seen in FIG. 5, prophylactic treatment of Compound 1, 1 and 3 mg/kg for 7 days significantly reduced the development of heat hyperalgesia compared vehicle treatment, at p<0.05 and p<0.0001 vs vehicle (at 0 time) respectively. After establishment of ES induced heat hyperalgesia, additional dose of Compound 1 on day 9, further enhanced the analgesic effect, achieving significance at 0.3 mg/kg, p<0.05, p<0.0001 and p<0.0001 post 2 hrs and p<0.001, p<0.0001 and p<0.0001 post 4 hrs for 0.3, 1 and 3 mg/kg respectively. Acute treatment of Compound 1 at 3 mg/kg also reversed the heat hyperalgesia, p<0.0001, post 2 and 4 hrs reproducing the earlier findings.

### Example 2c—Cold Thermal Allodynia (Acetone Test)

General considerations as given under Example 1c also apply here.

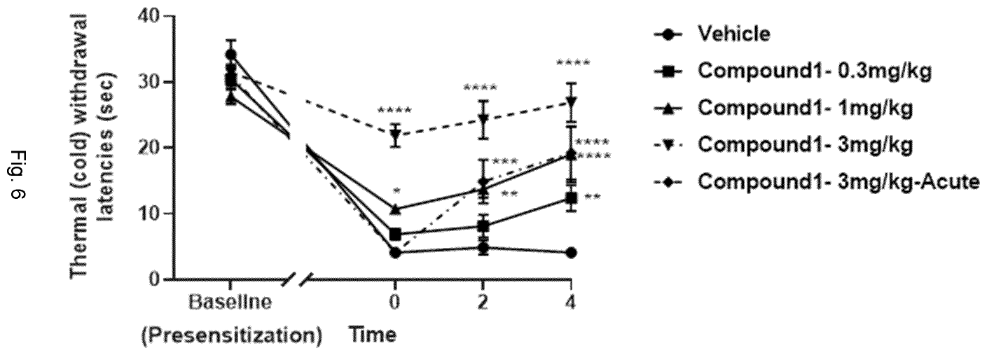

All rats displayed cold allodynia as evidenced by the robust decrease in cold pain threshold from baseline (average of Day 1 and Day 2 measurements) to Day 9 (0 time, p<0.0001 vs baseline). As seen in FIG. 6, prophylactic treatment of Compound 1, 1 and 3 mg/kg for 7 days significantly reduced the development of cold allodynia compared to vehicle treatment, p<0.05 and p<0.0001 vs vehicle (at 0 time) respectively. After establishment of ES induced cold allodynia, additional dose of Compound 1 on day 9, reversed the cold allodynia dose dependently, p>0.05, p<0.01 and p<0.0001 post 2 hrs and p<0.01, p<0.0001 and p<0.0001 post 4 hrs for 0.3, 1 and 3 mg/kg respectively. Acute treatment of Compound 1 at 3 mg/kg also reversed the mechanical allodynia, p<0.001 and p<0.0001, post 2 and 4 hrs respectively.

### Conclusion to Example 2

Direct electrical stimulation to depilated cheek area of rats produces the development of allodynia and hyperalgesia manifested as a marked reduction in mechanical and thermal pain thresholds after 22 hours of stimulation, despite no visible edema or swelling.

Prophylactic treatment with Compound 1 for 7 days, as a once daily dose, significantly reduced the development of ES-induced sensitization of the trigeminal sensory system as indicated by reversal of hypersensitivity to the mechanical and thermal stimulus. In particular, the data indicate that a once daily dose of 3 mg/kg of Compound 1 has the same analgesic effect as either a 10 mg/kg acute dose of Compound 1 or 30 mg/kg of Carbamazepine. In summary, it is possible to use Compound 1, which is not associated with any known side-effects, in a much lower dose compared to Carbamazepine, when administered prophylactically. These data support the potential effect of Compound 1 for use in prophylactic treatment of trigeminal neuralgia and other facial neuropathic pain disorders.

Furthermore, the data suggest that therapeutic agents such as Compound 1 may be used in treatment of postoperative pain originating from surgery and/or trauma to the human body. specifically, Compound 1 may conveniently be administered prophylactically in a time period before a subject undergoes surgery, in order to build nociceptive resistance in the subject, and the treatment continued post-surgery. Such a treatment would reduce the need of highly addictive post-surgery analgesics such as opioids and similar, which in addition to being addictive are also associated with tolerance build-up which limits the analgesic effect.

### References

Bendtsen L, Zakrzewska J M, Heinskou T B, Hodaie M, Leal P R L, Nurmikko T, Obermann M, Cruccu G, et al. (2020), Advances in diagnosis, classification, pathophysiology, and management of trigeminal neuralgia. Lancet Neurol 19:784-796.
Chaplan S R, Bach F W, Pogrel J W, Chung J M, Yaksh T L (1994), Quantitative assessment of tactile allodynia in the rat paw. J Neurosci Methods 53:55-63.
Luo D S, Zhang T, Zuo C X, Zuo Z F, Li H, Wu S X, Wang W, Li Y Q (2012), An animal model for trigeminal neuralgia by compression of the trigeminal nerve root. Pain Physician 15:187-196.
Maarbjerg S, Di Stefano G, Bendtsen L, Cruccu G (2017), Trigeminal neuralgia—diagnosis and treatment. Cephalalgia 37:648-657.
Obermann M (2019), Recent advances in understanding/managing trigeminal neuralgia. F1000Res 8.
Romero-Reyes M, Uyanik J M (2014), Orofacial pain management: current perspectives. J Pain Res 7:99-115.
Zeilhofer H U. et. al. (2009), GABAergic analgesia: new insights from mutant mice and subtype-selective agonists, Trends in pharmacological science, 30, 8, 397-402

1. A compound for use in the treatment of facial pain in a subject, wherein the compound is 2-(3-(3-(2,4-dimethoxypyrimidin-5-yl)phenyl)-3H-imidazo[4,5-b]pyridin-6-yl)propan-2-ol or a pharmaceutically acceptable salt thereof.

2. The compound for use according to claim 1, wherein the facial pain is orofacial pain and/or craniofacial pain.

3. The compound for use according to any one of the preceding claims, wherein the facial pain is trigeminal neuralgia (TN).

4. The compound for use according to any one of claims 1 to 2, wherein the facial pain is associated with migraine or headache.

5. The compound for use according to any one claims 1 to 2, wherein the pain is associated with atypical facial pain or mononeuropathies.

6. The compound for use according to any one of the preceding claims, wherein the subject is administered a dose in the range of 0.001-100 mg/kg perorally.

7. A compound for use in the treatment of postoperative pain in a subject, wherein the compound is 2-(3-(3-(2,4-dimethoxypyrimidin-5-yl)phenyl)-3H-imidazo[4,5-b]pyridin-6-yl)propan-2-ol or a pharmaceutically acceptable salt thereof.

8. The compound for use according to claim 7, wherein the postoperative pain is acute postoperative pain.

9. The compound for use according to claim 7, wherein the postoperative pain is chronic postoperative pain.

10. The compound for use according to claim 7, wherein the pain is chronic pain after surgery, such as after surgical procedures comprising spinal surgery, herniotomy, hysterectomy, amputation, thoracotomy, breast surgery, and arthroplasty.

11. The compound for use according to any one of claims 7 to 10, wherein the treatment of the subject is initiated more than 2 days prior to the subject undergoing surgery.

12. The compound for use according to any one of claims 7 to 11, wherein the treatment is initiated more than 2 days, such as 3, such as 4, such as 5, such as 6, such as 7, such as 10, such as 20, such as 30 days prior to the subject undergoing surgery.

13. The compound for use according to any one of claims 7 to 12, wherein the subject is administered a dose in the range of 0.001-100 mg/kg perorally.

14. A method for treatment of facial pain and/or postoperative pain comprising administration of 2-(3-(3-(2,4-dimethoxypyrimidin-5-yl)phenyl)-3H-imidazo[4,5-b]pyridin-6-yl)propan-2-ol, or a pharmaceutically acceptable salt thereof, to a subject in need thereof.

15. Use of 2-(3-(3-(2,4-dimethoxypyrimidin-5-yl)phenyl)-3H-imidazo[4,5-b]pyridin-6-yl)propan-2-ol, or a pharmaceutically acceptable salt thereof, for the manufacture of a medicament for the treatment of facial pain and/or postoperative pain.
